(19) United States
(12) Patent Application Publication
(10) Pub. No.: US 2020/0038451 A1
(43) Pub. Date: Feb. 6, 2020

(54) STEM CELLS AS AN INDIVIDUALIZED MATERNAL THERAPY FOR PREVENTION OF PREMATURITY

(71) Applicants: THE JOHNS HOPKINS UNIVERSITY, Baltimore, MD (US); KENNEDY KRIEGER INSTITUTE, INC., Baltimore, MD (US)

(72) Inventors: Irina Burd, Silver Spring, MD (US); Karin Blakemore, Baltimore, MD (US); Michael V. Johnston, Baltimore, MD (US); Ali S. Fatemi, Bethesda, MD (US)

(21) Appl. No.: 16/589,220

(22) Filed: Oct. 1, 2019

Related U.S. Application Data
(63) Continuation of application No. 14/401,170, filed on Nov. 14, 2014, now abandoned.

(51) Int. Cl. A61K 35/28; C12N 5/0775; A61K 9/00

(52) U.S. Cl. CPC A61K 35/28; A61K 9/0034; C12N 5/0667

(57) ABSTRACT

The present invention relates to the field of premature birth. More specifically, the present invention provides methods and compositions useful for preventing premature birth. In one embodiment, a method for preventing preterm birth in a patient comprises the step of administering to the patient an effective amount of autologous mesenchymal stem cells (MSCs) during the first or second trimester.

### CROSS-REFERENCE TO RELATED APPLICATIONS

This application is a Continuation of U.S. patent application Ser. 14/401,170, filed Nov. 14, 2014, which is a 35 U.S.C. § 371 U.S. national entry of International Application PCT/US2013/041287, having an international filing date of May 16, 2013, which claims the benefit of U.S. Provisional Application No. 61/647,739, filed May 16, 2012, the content of each of the aforementioned applications is herein incorporated by reference in their entirety.

### STATEMENT OF GOVERNMENTAL INTEREST

This invention was made with government support under grant no. NS063956 awarded by the National Institutes of Health. The government has certain rights in the invention.

### FIELD OF THE INVENTION

The present invention relates to the field of premature birth. More specifically, the present invention provides methods and compositions useful for preventing premature birth.

### BACKGROUND OF THE INVENTION

In the United States, approximately 12% of all live births are preterm. Although mechanisms underlying spontaneous preterm birth are not well understood, intrauterine inflammation has been associated with majority of the cases. Intrauterine inflammation represents an abnormal polarization of Th1/Th2 axes towards Th1, and a failed host response. The presence of intrauterine inflammation has been linked to a devastating spectrum of neurobehavioral disorders in these children ranging from learning disability to motor deficits such as cerebral palsy. Rescuing a failed host response may prove to decrease the rate of preterm birth and decrease prematurity-related morbidity worldwide.

### SUMMARY OF THE INVENTION

The present invention is based, at least in part, on the discovery that mesenchymal stem cells (MSCs) can be used to prevent premature birth. As described herein, MSCs are able to keep maternal and fetal immune systems in check, after exposure to intrauterine inflammation, and with that decrease preterm birth rate and perinatal brain injury. Pretreatment with MSCs appears to immunomodulate maternal and fetal response to intrauterine inflammation. Rescued host response was associated with decreased preterm birth and a decrease in fetal brain injury. The present invention is the first to suggest that MSCs harvested from women with history of preterm birth may have a potential to serve as a personalized cell therapy “vaccine” in their future pregnancy.

Accordingly, in one aspect, the present invention provides methods and composition useful for preventing preterm birth. In one embodiment, a method for preventing preterm birth in a patient comprises the step of administering to the patient an effective amount of autologous mesenchymal stem cells (MSCs) during the first or second trimester. In certain embodiments, the autologous MSCs are derived from adipose tissue. In other embodiments, the autologous MSCs are derived from bone marrow. In particular embodiments, the effective amount of autologous MSCs comprises about 2×10⁵−1×10⁶ cells/kg. In a specific embodiment, the MSCs are administered intravenously. In another embodiment, the MSCs are administered via intrauterine injection. In certain embodiments, the patient has a history of preterm birth. In particular embodiments, the MSCs are collected prior to the pregnancy.

The present invention also provides methods method for preventing pre-term birth in a patient comprising the step of administering to the patient an effective amount of autologous adipose tissue-derived MSCs during the first or second trimester. In some embodiments, the effective amount of autologous MSCs comprises about 2×10⁵−1×10⁶ cells/kg. The MSCs can be administered intravenously or via intrauterine injection. In certain instances, the patient has a history of preterm birth. In particular embodiments, the MSCs are collected prior to the pregnancy.

In another embodiment, a method for preventing pre-term birth in a patient comprises the steps of (a) collecting adipose tissue from the patient prior to pregnancy; (b) processing the tissue to generate substantially purified MSCs; and (c) administering the MSCs to the patient during the first or second trimester of a subsequent pregnancy. In certain embodiments, the administered amount of autologous MSCs comprises about 2×10⁵−1×10⁶ cells/kg. The MSCs are administered intravenously or via intrauterine injection. In certain instances, the patient has a history of preterm birth.

The present invention also provides a method for preventing intrauterine inflammation in a pregnant woman with a history of preterm birth comprising the step of administering an effective amount of autologous MSCs prior to intrauterine inflammation. In another embodiment, a method for preventing pre-term birth in a patient comprises the steps of (a) collecting adipose tissue from the patient during the first or second trimester; (b) processing the tissue to generate substantially purified MSCs; and (c) administering the MSCs to the patient. In a specific embodiment, steps (a)-(c) are performed consecutively while the patient waits.

In particular embodiments, the amount of autologous MSCs administered to the patient comprises about 1×10⁵−1×10⁸ cells/kg. More specifically, the number of MSCs may comprise about 2×10⁵−5×10⁷, about 3×10⁵−3×10⁷, about 4×10⁵−2×10⁷, about 5×10⁵−1×10⁷, about 6×10⁵−9×10⁶, about 7×10⁵−8×10⁷, about 8×10⁵−7×10⁷, and so on.

In certain embodiments, the autologous MSCs are derived from adipose tissue. In other embodiments, the autologous MSCs are derived from bone marrow. In specific embodiments, the tissue can be manipulated or processed to result in substantially purified MSCs. In a more specific embodiment, the MSC are at least 50%, least 55%, at least 60%, at least 65%, at least 70%, at least 75%, at least 80%, at least 85%, at least 90% or at least 95% free of other components from which the MSCs were first collected (e.g., harvested from adipose tissue or bone marrow).

### BRIEF DESCRIPTION OF THE FIG.S

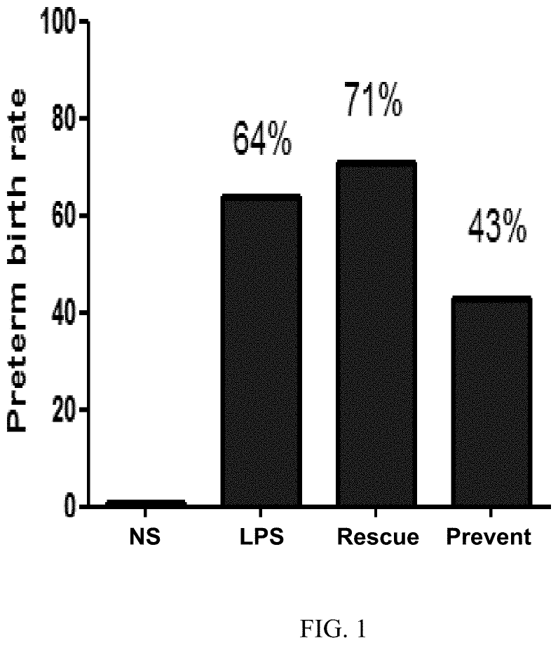

FIG. 1. Maternally administered human adipose derived mesenchymal stem cells (MSC) appear to modulate maternal response to intrauterine inflammation and decrease preterm birth rate in the murine model. Pretreatment (Prevent) with adipose derived MSCs, but not post-treatment (Rescue), significantly decreased the rate of preterm birth (p<0.01, chi square) by 21% (n=56 dams divided between 4 groups). NS, normal saline (negative control); LPS, lipopolysaccharide (positive control; exposure to in utero inflammation).

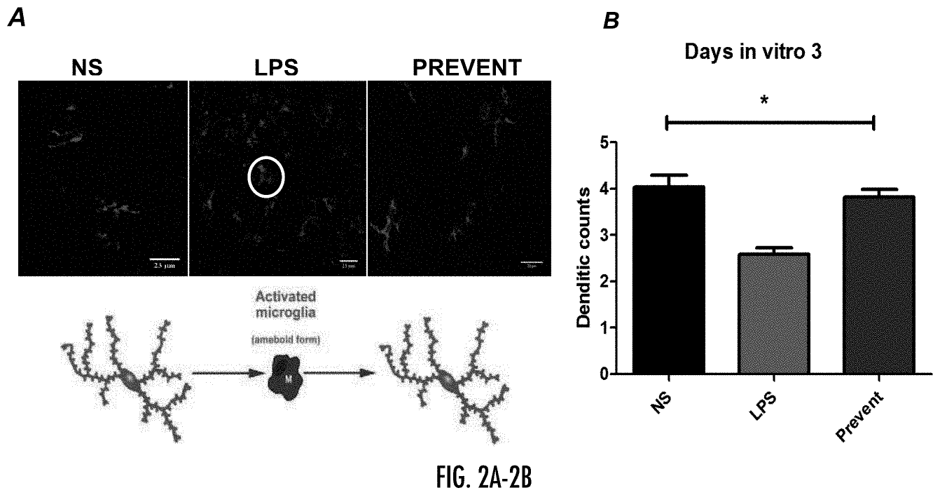

FIG. 2. Maternally administered adipose derived MSCs decreased perinatal brain injury. A, Immunohistochemical evaluation of fetal brain in periventricular area demonstrated activation of migroglia (Iba1 stain), following in utero LPS exposure (middle panel; circle). In pretreatment group (PREVENT), MSC administration prior to LPS exposure, prevented microglial activation, as the structures were similar to negative control group (NS) microglia. B, Primary cortical cultures of fetal neurons were examined with immunocytochemistry (MAP2 an NF200) for neurotoxicity at days in vitro 3 (dendritic counts). As expected, LPS exposure decreased number of dendrites as compared to control, normal saline (NS) (P<0.05, SNK test; red bar). In the pretreatment group (Prevent; blue bar), the number of dendrites was significantly increased as compared to LPS-exposed group P<0.05; SNK test) and was similar to control (NS; P>0.05, SNK test; black bar). *P<0.05, One-way ANOVA, Student-Newman-Keuls (SNK) test was used for multiple comparisons. NS, normal saline (negative control); LPS, lipopolysaccharide (positive control; exposure to in utero inflammation).

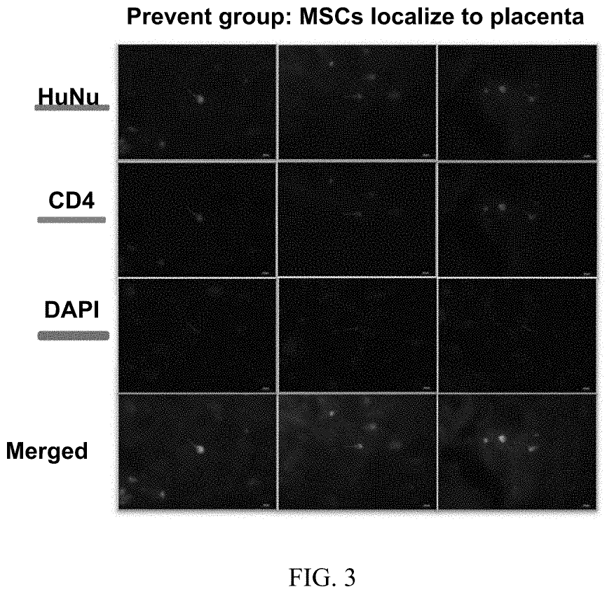

FIG. 3. Maternally administered human adipose derived mesenchymal stem cells (MSCs) in pretreatment group (PREVENT) localized to murine placenta. Immunohistochemistry of murine placentas revealed successful double staining specific for human nucleus (HuNu) and CD44, specific for MSCs. DAPI stain localizes to DNA material. All 3 panels demonstrate that human MSCs administered intraperitoneally localized to murine placenta (merged images in all 3 columns). MSCs were not detected in any of the fetal compartments within 24 hours of administration (data not shown).

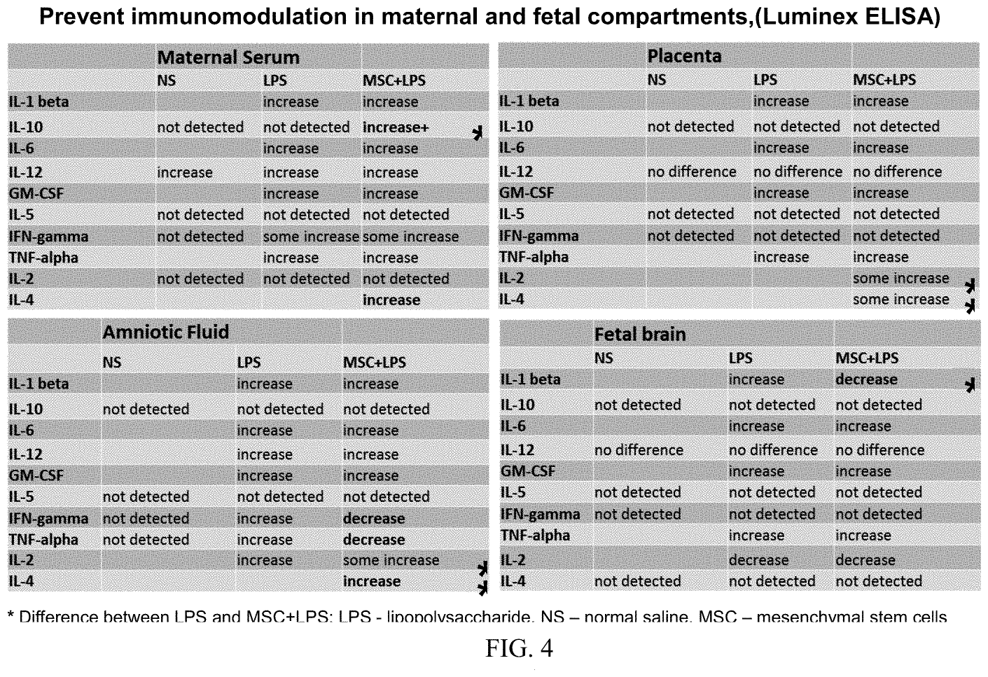

FIG. 4. Table showing pretreatment immunomodulation in maternal and fetal compartments.

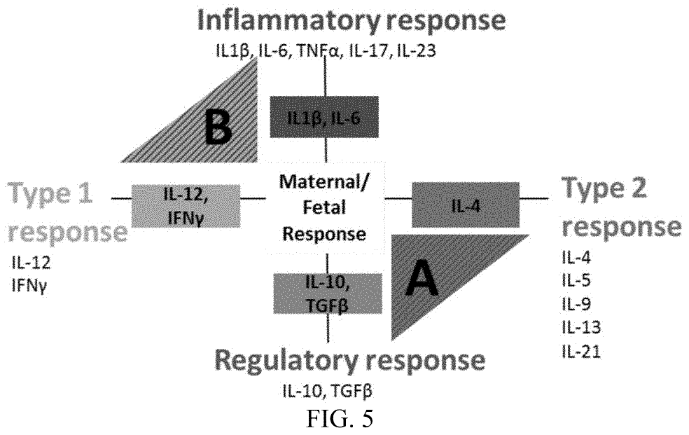

FIG. 5. Normal and successful pregnancy is associated with and requires polarization toward T helper 2 (Type 2) response (zone A). Inflammation within uterus triggers an opposite shift toward T helper 1 (Type 1) response (also known as rejection; zone B). The immunomodulatory effects of MSCs on different cellular components of innate and adaptive immunity include: inhibition of pro-inflammatory cytokine secretion and decrease in cytotoxic potential of natural killer cells. They are also known to modulate macrophage response to inflammation by increasing secretion if IL-10 from macrophages and deceasing TNFα and IL-6 secretion. Maternal pretreatment with MSCs will shift the axis of inflammation-associate cytokine response in maternal/fetal compartments toward a normal response in pregnancy; zone A.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth.

Unless defined otherwise, all technical and scientific terms used herein have the same meaning as commonly understood by one of ordinary skill in the art to which this invention belongs. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

“Adipose” refers to any fat tissue. The adipose tissue may be brown or white adipose tissue. The adipose may be mesenchymal or stromal. In certain embodiments, the adipose tissue is subcutaneous white adipose tissue. The adipose tissue may be from any organism having fat tissue. In most embodiments, the adipose tissue is mammalian, most preferably the adipose tissue is human. A convenient source of human adipose tissue is that derived from liposuction surgery or other surgery. However, the source of adipose tissue or the method of isolation of adipose tissue is not critical to the invention.

As used herein, the term “adipose cell” is used to refer to any type of adipose tissue, including an undifferentiated adipose-derived adult stem cell and a differentiated adipose-derived adult stem cell.

The term “adipose tissue-derived cell” herein refers to a cell that originates from adipose tissue, preferably from the blood vessels contained therein. The initial cell population isolated from adipose tissue is a heterogeneous cell population including, but not limited to stromal or mesenchymal vascular fraction (SVF) or (MVF) cell.

As used herein, the term “adipose-derived stem cell” (“ADSC” or “ASC”) refers to stromal or mesenchymal cells that originate from blood vessels found in adipose tissue which can serve as stem cell-like precursors to a variety of different cell types such as but not limited to adipocytes, osteocytes, chondrocytes, muscle and neuronal/glial cell lineages. Adipose-derived stem cells make up a subset population derived from adipose tissue which can be separated from other components of the adipose tissue using standard culturing procedures or other methods disclosed herein. In addition, adipose-derived adult stem cells can be isolated from a mixture of cells using cell surface markers. The term ADSC or ADC thus includes or comprises MSCs.

The term “mesenchymal stem cell” (“MSC”) refers to an adherent stroma cell, for example from a biological sample such as adipose tissue, bone marrow or umbilical cord blood, isolated by methods such as those provided herein and by U.S. Pat. Nos. 7,060,494; 5,965,436; 5,908,784; 5,906,934; 5,858,390; 5,827,735; 5,654,186; and 5,486,359. Such cells have been characterized by being multipotent stem cells that have the capacity to differentiate into osteoblasts, adipocytes and chondrocytes in vitro and express the surface antigens including CD105, CD73 and CD90, but not CD45 or CD34. See Dominici et al, 8 CYTOTHERAPY 315-17 (2007).

As used herein the phrase “mesenchymal or stromal vascular fraction” refers to a cell fraction derived from blood vessels found in adipose tissue that comprises different cell types including mesenchymal stem cells, hematopoietic cells, hematopoietic stem cells, platelets, Kupffer cells, osteoclasts, megakaryocytes, granulocytes, NK cells, endothelial precursor or progenitor cells, CD34+ cells or mesenchymal stem cells, (typically found in umbilical cord), CD29+ cells, CD166+ cells, Thy-1+ or CD90+ stem cells, CD44+ cells, immune cells such as monocytes, leukocytes, lymphocytes, B and T cells, NK cells, macrophages, neutrophil leukocytes, neutrophils, neutrophil granulocytes, and the like including immune and other cells that express one or more of the following markers: CD3, CD14 (macrophage marker), CD19, CD20 (B cell marker), CD29 (integrin unit), CD31 (endothelial, platelet, macrophage, Kupffer cell, dendritic cell, granulocyte, T/NK cells, lymphocytes, megakaryocytes, osteoclasts, neutrophils, et al.), CD44 (Hyaluronic acid receptor), CD45 (B and T cell marker), C56, CD73 (lymphocyte differentiation marker), CD105 et al. Also, it includes cells expressing any of the markers or any combination thereof disclosed in this application.

Adipose tissue can be obtained or collected by any method known to a person of ordinary skill in the art. For example, adipose tissue may be removed from a patient by liposuction (syringe or power assisted) or by lipectomy, e.g., suction-assisted lipoplasty, ultrasound-assisted lipoplasty, and excisional lipectomy or combinations thereof. The adipose tissue is removed and collected and may be processed in accordance with any of the embodiments of a system of the invention described herein. The amount of tissue collected depends on numerous factors, including the body mass index and age of the donor, the time available for collection, the availability of accessible adipose tissue harvest sites, concomitant and pre-existing medications and conditions (such as anticoagulant therapy), and the clinical purpose for which the tissue is being collected.

After the adipose tissue is processed, the resulting regenerative cells are substantially free from mature adipocytes and connective tissue. Accordingly, utilizing a system known in the art generates a heterogeneous plurality of adipose derived regenerative cells which may be used for research and/or the therapeutic purposes described herein. In certain embodiments, the cells are suitable for placement or re-infusion within the body of a recipient. In other embodiments, the cells may be used for research, e.g., the cells can be used to establish stem or progenitor cell lines which can survive for extended periods of time and be used for further study.

As used herein, the terms “administering,” “introducing,” “delivering,” “placement” and “transplanting” are used interchangeably herein and refer to the introduction of the cells of the present invention into a subject or patient. In certain embodiments, the terms mean providing to a human patient a pharmaceutical preparation containing mesenchymal stem cells (e.g., adipose-tissue derived MSCs), optionally in the form of MSC spheres or foci, or their progeny or derivatives in a suitable formulation. The preferred method of administration can vary depending on various factors, e.g., the components of the pharmaceutical preparation, etc. and specifically include intravenous or intrauterine injection. In other embodiments, the compositions of the present invention may be administered by any particular route of administration including, but not limited to parenteral, subcutaneous, intramuscular, intravenous, intrarticular, intraabdominal, intracavitary, intracervical, intragastric, intrapelvic, and intraperitoneal. The cells can be administered by any appropriate route which results in delivery to a desired location in the subject where at least a portion of the cells or components of the cells remain viable.

The number of cells administered to the patient can vary. In particular embodiments, the amount of autologous MSCs administered to the patient comprises about 1×10⁵−1×10⁸ cells/kg. More specifically, the number of MSCs may comprise about 2×10⁵−5×10⁷, about 3×10⁵−3×10⁷, about 4×10⁵−2×10⁷, about 5×10⁵−1×10⁷, about 6×10⁵−9×10⁶, about 7×10⁵−8×10⁷, about 8×10⁵−7×10⁷, and so on. In a specific embodiment, the amount of MSCs administered to the patient comprises about 2×10⁵−1×10⁶ cells/kg.

The term “autologous” means derived from the same individual or involving one individual as both donor and recipient.

The term “cell culture” means grown outside of the body in a dish, flask, or other container in the presence of growth media. Cell culture can be performed with transformed or immortalized cell lines. Cell culture can also be performed with “primary cells” removed from an animal, such as a mammal, and are not transformed or immortalized. Primary cells can be dividing or non-dividing cells. For example, the cells can be bone marrow cells, umbilical cord blood cells, or mesenchymal stem cells.

The term “effective amount” refers to an amount sufficient to effect beneficial or desired clinical or biochemical results. An effective amount can be administered one or more times. For purposes of this invention, an effective amount is the amount of MSCs to prevent preterm birth.

The terms “obtaining,” “harvesting,” and “collecting” as in obtaining, harvest or collecting a cell, respectively, refer to purchasing, synthesizing, or otherwise procuring a cell. Cells can be obtained, for example, from an animal including human and non-human animals. Cells can also be obtained from cell and tissue repositories. In specific embodiments, cells are obtained, harvested or collected from a patient, processed and subsequently administered back to the patient to prevent premature birth.

As used herein, the term “processed lipoaspirate” refers to adipose tissue that has been processed to separate the active cellular component (e.g., the component containing regenerative/stem cells) from the mature adipocytes and connective tissue. This fraction is referred to herein as “adipose-derived cells” or “ADC.” Thus, ADC comprises stem cells (e.g., MSCs). MSCs derived from adipose tissue are referred to as adipose-derived MSCs. Typically, ADC refers to the pellet of regenerative cells obtained by washing and separating and concentrating the cells from the adipose tissue. The pellet is typically obtained by centrifuging a suspension of cells so that the cells aggregate at the bottom of a centrifuge chamber or cell concentrator.

By “substantially purified” or “substantially free” is meant that the desired cells (e.g., MSCs) are enriched by at least 30%, at least 35%, at least 40%, at least 45%, at least 50%, at least 55%, at least 60%, at least 65%, at least 70%, at least 75%, at least 80%, at least 85%, at least 90% or at least 95%. In a specific embodiments, adipose tissue can be manipulated or processed to result in substantially purified MSCs. In a more specific embodiment, the MSC are at least 50%, least 55%, at least 60%, at least 65%, at least 70%, at least 75%, at least 80%, at least 85%, at least 90% or at least 95% free of other components from which the MSCs were first collected (e.g., adipose tissue).

By “treatment” is meant an approach for obtaining beneficial or desired clinical results. For the purposes of this invention, beneficial or desired clinical results include, but are not limited to, alleviation of symptoms, diminishment of extent of disease, stabilization (i.e., not worsening) of a state of disease, delay or slowing of disease progression, amelioration or palliation of the disease state, and remission (whether partial or total), whether detectable or undetectable. In certain embodiments, the term refers to the prevention of preterm birth. “Treatment” refers to both therapeutic treatment and prophylactic or preventative measures.

MSCs represent a promising tool for cell therapy. They are currently being tested in U.S. FDA-approved clinical trials for myocardial infarction, stroke, limb ischemia, graft-versus-host disease, and autoimmune disorders. Furthermore, MSCs have been tested for the treatment of neurodegenerative diseases and are known to regulate inflammation and promote endogenous neuronal growth, decrease apoptosis, and encourage synaptic connection from damaged neurons. MSCs are known to reprogram macrophages to produce IL-10 and to counteract inflammation. The present inventors have discovered that MSCs are able to keep the maternal and fetal immune system in check after exposure to intrauterine inflammation (FIG. 5).

Accordingly, in some embodiments, the mesenchymal stem cells are derived from adipose tissue, in particular liposuctioned fat, bone marrow, blood, dental pulp, cornea, undifferentiated cell lineages such as undifferentiated fibroblasts, and combinations thereof. In particular embodiments, the MSCs are adipose tissue-derived mesenchymal stem cells, due to their easy obtention (either from liposuction or lipectomy), a low donor-site morbidity and a high cell yield. In other embodiments, MSCs are derived from bone marrow.

The Celution® System (Cytori Therapeutics, Inc. (San Diego, Calif.)) is one of several medical devices that enable access to adult adipose-derived stem cells (ADSCs) by automating and standardizing the extraction, washing, and concentration of a patient's own ADSCs for present and future clinical use. See U.S. Pat. Nos. 8,337,834; 8,246,947; 8,136,276; 8,119,121; 7,771,716; 7,687,059; 7,585,670; 7,473,420; 7,429,488; and 7,390,484.

Another medical device useful in the present invention is the IntelliCell™ process developed by IntelliCell Biosciences, Inc. (New York, N.Y.). See U.S. Pat. No. 8,440,440; and U.S. patent application Ser. No. 13/745,367. Briefly, the patient visiting the clinic receives a mini-liposuction procedure under local anesthetic, and the physician remove about 60 ccs of adipose (fat) tissue from the abdomen. Adipose tissue is primarily composed of the adipocyte tissue (80%) and a network of mostly capillaries that surround the adipocytes.

The IntelliCell™ process uses ultrasound to separate the network of capillaries from the adipocytes. In a closed sterile process that is very similar to obtaining cells from bone marrow, the vascular tissue after it has been separated from the adipocytes, is washed in a sterile area and placed in a centrifuge and spun at low levels for several minutes. The actual fat tissue that was obtained via the liposuction procedure is discarded. The autologous vascular cells drop to the bottom of the collection container and are prepared for quality testing. IntelliCell™ uses a flow cytometer to check each sample for cell viability and the cell count for each patient. The entire process takes about 1 hour to complete. The cells are then returned to the physician and the patient treatment can begin. Some of the cells are placed into an IV drip bag for administration. The IV treatment takes about 20 minutes. Alternatively, the cells can also be placed locally (e.g., intrauterine injection).

The present invention utilizes systems and methods for separating and concentrating regenerative cells, e.g., stem cells and/or progenitor cells, from a wide variety of tissues including, but not limited to, adipose, bone marrow, blood, skin, muscle, liver, connective tissue, fascia, brain and other nervous system tissues, blood vessels, and other soft or liquid tissues or tissue components or tissue mixtures (e.g., a mixture of tissues including skin, blood vessels, adipose, and connective tissue). In certain embodiments, the system separates and concentrates MSCs from adipose tissue. In another embodiment, the system is automated such that the entire method may be performed with minimal user intervention or expertise. In a particular embodiment, the MSCs obtained using the systems and methods of the present invention are suitable for direct placement into a subject with a history of preterm birth from whom the tissue was extracted.

In particular embodiments, the entire procedure from tissue extraction through separating, concentrating and placement of the cells (comprising MSCs) into the subject would all be performed in the same facility, indeed, even within the same room of the patient undergoing the procedure. The cells may be used in a relatively short time period after extraction and concentration. For example, the cells may be ready for use in about one hour from the harvesting of tissue from a patient, and in certain situations, may be ready for use in about 10 to 40 minutes from the harvesting of the tissue. In a specific embodiment, the cells may be ready to use in about 20 minutes from the harvesting of tissue. The entire length of the procedure from extraction through separating and concentrating may vary depending on a number of factors, including patient profile, type of tissue being harvested and the amount of cells required for a given therapeutic application. The cells may also be placed into the recipient in combination with other cells, tissue, tissue fragments, scaffolds or other stimulators of cell growth and/or differentiation in the context of a single operative procedure with the intention of deriving a therapeutic, structural, or cosmetic benefit to the recipient. It is understood that any further manipulation of the cells beyond the separating and concentrating phase of the system will require additional time commensurate with the manner of such manipulation.

During the processing, one or more additives may be used as needed to enhance the results. Some examples of additives include agents that optimize washing and disaggregation, additives that enhance the viability of the active cell population during processing, anti-microbial agents (e.g., antibiotics), additives that lyse adipocytes and/or red blood cells, or additives that enrich for cell populations of interest (by differential adherence to solid phase moieties or to otherwise promote the substantial reduction or enrichment of cell populations). For example, to obtain a homogenous cell population, any suitable method for separating and concentrating the particular cell type (e.g., MSCs) may be employed, such as the use of cell-specific antibodies that recognize and bind antigens present on, for example, stem cells or progenitor cells, e.g., MSCs. These include both positive selection (selecting the target cells), negative selection (selective removal of unwanted cells), or combinations thereof. Intracellular markers such as enzymes may also be used in selection using molecules which fluoresce when acted upon by specific enzymes. In addition, a solid phase material with adhesive properties selected to allow for differential adherence and/or elution of a particular population of regenerative cells within the final cell pellet could be inserted into the system.

An alternate embodiment of this differential adherence approach would include use of antibodies and/or combinations of antibodies recognizing surface molecules differentially expressed on target regenerative cells and unwanted cells. Selection on the basis of expression of specific cell surface markers (or combinations thereof) is another commonly applied technique in which antibodies are attached (directly or indirectly) to a solid phase support structure. In another embodiment the cell pellet could be re-suspended, layered over (or under) a fluid material formed into a continuous or discontinuous density gradient and placed in a centrifuge for separation of cell populations on the basis of cell density. In a similar embodiment, continuous flow approaches such as apheresis, and elutriation (with or without counter-current) may also be employed.

Other examples of additives may include additional biological or structural components, such as cell differentiation factors, growth promoters, immunosuppressive agents, medical devices, or any combinations thereof. For example, other cells, tissue, tissue fragments, growth factors such as VEGF and other known angiogenic or arteriogenic growth factors, biologically active or inert compounds, resorbable scaffolds, or other additives intended to enhance the delivery, efficacy, tolerability, or function of the population of cells may be added.

The cell population may also be modified by insertion of DNA or by placement in a cell culture system (as described herein or known in the art) in such a way as to change, enhance, or supplement the function of the cells for derivation of a structural or therapeutic purpose. For example, gene transfer techniques for stem cells are known by persons of ordinary skill in the art and may include viral transfection techniques, and more specifically, adeno-associated virus gene transfer techniques. Non-viral based techniques may also be performed. A gene encoding one or more cellular differentiating factors, e.g., a growth factor(s) or a cytokine(s), could also be added. Examples of various cell differentiation agents are disclosed in Gimble et al., 1995; Lennon et al., 1995; Majumdar et al., 1998; Caplan and Goldberg, 1999; Ohgushi and Caplan, 1999; Pittenger et al., 1999; Caplan and Bruder, 2001; Fukuda, 2001; Worster et al., 2001; Zuk et al., 2001. Genes encoding anti-apoptotic factors or agents could also be added. Addition of the gene (or combination of genes) could be by any technology known in the art including but not limited to adenoviral transduction, gene guns, liposome-mediated transduction, and retrovirus or lentivirus-mediated transduction, plasmid, adeno-associated virus. These cells could then be implanted along with a carrier material bearing gene delivery vehicle capable of releasing and/or presenting genes to the cells over time such that transduction can continue or be initiated in situ.

When the cells and/or tissue containing the cells are administered to a patient other than the patient from whom the cells and/or tissue were obtained, one or more immunosuppressive agents may be administered to the patient receiving the cells and/or tissue to reduce, and preferably prevent, rejection of the transplant. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. patent Pub. No. 20020182211. A preferred immunosuppressive agent is cyclosporine A. Other examples include myophenylate mofetil, rapamicin, and anti-thymocyte globulin. In one embodiment, the immunosuppressive drug is administered with at least one other therapeutic agent. The immunosuppressive drug is administered in a formulation which is compatible with the route of administration and is administered to a subject at a dosage sufficient to achieve the desired therapeutic effect. In another embodiment, the immunosuppressive drug is administered transiently for a sufficient time to induce tolerance to the regenerative cells of the invention.

In all of the foregoing embodiments, at least a portion of the separated and concentrated regenerative cells may be cryopreserved. The cells can be used at a later time, prior to/during subsequent pregnancies to prevent preterm birth. In such embodiments, the cells are collected between pregnancies from “at-risk” patients (history of pre-term birth), and the autologous MSCs would be infused in a future pregnancy.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions.

### Example 1: Immunomodulatory Therapy for Preterm Birth and Prematurity Related Morbidity

Objective: Using a mouse model of intrauterine inflammation and preterm birth, we have demonstrated that exposure to inflammation induces perinatal brain injury. Adipose tissue derived mesenchymal stem cells have been shown to exhibit immunomodulary effects in other inflammatory conditions. We hypothesized that treatment with human adipose tissue derived mesenchymal stem cells (hMSC) may decrease the rate of preterm birth and perinatal brain injury through an increase in the anti-inflammatory milieu.

Study Design: A mouse model of intrauterine inflammation and preterm birth was utilized (n=56 dams in 4 treatment groups) at E17 of gestation (preterm), with the following groups: 1) control—normal saline (NS); 2) intrauterine (IU) inflammation (LPS); 3) IU LPS+intraperitoneal (IP) hMSC 30 min after the onset of inflammation (Rescue); and 4) intrauterine LPS+IP hMSC 15 hrs prior to the onset of inflammation (Prevent). Maternal serum (MS), amniotic fluid (AF) and fetal and neonatal brains were collected. Luminex Multiplex ELISAs were performed for protein levels of pro-inflammatory and anti-inflammatory cytokines. Fetal brains were processed for primary cortical cultures of fetal neurons and molecular studies. Primary culture of fetal neurons was examined with immunofluorescence (MAP2 and NF200) for morphology, and neurotoxicity. Statistical analysis was performed with One way ANOVA, ANOVA on ranks and chi square where appropriate.

Results: Pretreatment with hMSC but not the post-treatment, significantly decreased the rate of preterm birth (p<0.01) by 21%. Pretreatment was associated with increase in IL-10 in MS (p<0.05) and IL-4 in AF (p<0.05); decrease in IL1β cytokine expression in fetal and neonatal brains, and fetal neurotoxicity (p<0.05).

Conclusion: Maternally administered adipose derived mesenchymal stem cells (MSC) appear to modulate maternal and fetal response to intrauterine inflammation in a murine model.

We claim:

1. A method for preventing preterm birth in a patient comprising the step of administering to the patient an effective amount of autologous mesenchymal stem cells (MSCs) during the first or second trimester

2. The method of claim 1, wherein the autologous MSCs are derived from adipose tissue.

3. The method of claim 1, wherein the autologous MSCs are derived from bone marrow.

4. The method of claim 1, wherein the effective amount of autologous MSCs comprises about 2×10⁵−1×10⁶ cells/kg.

5. The method of claim 1, wherein the MSCs are administered intravenously.

6. The method of claim 1, wherein the MSCs are administered via intrauterine injection.

7. The method of claim 1, wherein the patient has a history of preterm birth.

8. The method of claim 1, wherein the MSCs are collected prior to the pregnancy.

9. A method for preventing pre-term birth in a patient comprising the step of administering to the patient an effective amount of autologous adipose tissue-derived MSCs during the first or second trimester.

10. The method of claim 9, wherein the effective amount of autologous MSCs comprises about 2×10⁵−1×10⁶ cells/kg.

11. The method of claim 9, wherein the MSCs are administered intravenously.

12. The method of claim 9, wherein the MSCs are administered via intrauterine injection.

13. The method of claim 9, wherein the patient has a history of preterm birth.

14. The method of claim 9, wherein the MSCs are collected prior to the pregnancy.

15. A method for preventing pre-term birth in a patient comprising the steps of:
a. collecting adipose tissue from the patient prior to pregnancy;
b. processing the tissue to generate substantially purified MSCs; and
c. administering the MSCs to the patient during the first or second trimester of a subsequent pregnancy.

16. The method of claim 15 wherein the administered amount of autologous MSCs comprises about 2×10⁵−1×10⁶ cells/kg.

17. The method of claim 15, wherein the MSCs are administered intravenously.

18. The method of claim 15, wherein the MSCs are administered via intrauterine injection.

19. The method of claim 15, wherein the patient has a history of preterm birth.

20. A method for preventing intrauterine inflammation in a pregnant woman with a history of preterm birth comprising the step of administering an effective amount of autologous MSCs prior to intrauterine inflammation.

21. The method of claim 20 wherein the administered amount of autologous MSCs comprises about 2×10⁵−1×10⁶ cells/kg.

22. The method of claim 20, wherein the MSCs are administered intravenously.

23. The method of claim 20, wherein the MSCs are administered via intrauterine injection.

24. The method of claim 20, wherein the MSCs are collected prior to the pregnancy.

25. A method for preventing pre-term birth in a patient comprising the steps of:
a. collecting adipose tissue from the patient during the first or second trimester;
b. processing the tissue to generate substantially purified MSCs; and
c. administering the MSCs to the patient.

26. The method of claim 25, wherein steps (a)-(c) are performed consecutively while the patient waits.

27. The method of claim 25 wherein the administered amount of autologous MSCs comprises about 2×10⁵−1×10⁶ cells/kg.

28. The method of claim 25, wherein the MSCs are administered intravenously.

29. The method of claim 25, wherein the MSCs are administered via intrauterine injection.

30. The method of claim 25, wherein the patient has a history of preterm birth.
